(19) United States
(12) Patent Application Publication
(10) Pub. No.: US 2018/0156777 A1
(43) Pub. Date: Jun. 7, 2018

(54) METHOD AND APPARATUS FOR HIGH-RESOLUTION IMAGING OF CELL-CELL COMMUNICATION

(71) Applicant: The Methodist Hospital, Houston, TX (US)

(72) Inventors: Lidong QIN, Houston, TX (US); Joon Hee JANG, Houston, TX (US); Dongfang LIU, Houston, TX (US)

(21) Appl. No.: 15/569,999

(22) PCT Filed: May 2, 2016
(86) PCT No.: PCT/US2016/030408

§ 371 Date: Oct. 27, 2017

Related U.S. Application Data
(60) Provisional application No. 62/243,257, filed on Oct. 19, 2015.
(60) Provisional application No. 62/155,762, filed on May 1, 2015.

(51) Int. Cl. G01N 33/50; G01N 1/31; G01N 1/20; G01N 21/64; G01N 33/487; B01L 3/00

(52) U.S. Cl. CPC G01N 33/5005; G01N 1/312; G01N 1/20; G01N 21/6458; G01N 21/6428; B01L 2200/025; B01L 3/502761; B01L 3/502715; G01N 33/5014; B01L 2300/0858; B01L 2300/041; G01N 33/487

(57) ABSTRACT

A vertical cell pairing (VCP) system comprising: a substrate having a top surface; at least one micropit formed in the top surface of the substrate, the at least one micropit being sized to seat a cell; and at least one microtrap positioned adjacent the top surface of the substrate, the at least one microtrap comprising a body having a vertical slot, wherein the vertical slot has an opening and an exit, the vertical slot being sized to pass fluid therethrough but to prevent a cell from passing therethrough; wherein the at least one microtrap is disposed relative to the at least one micropit so that a cell seated in the at least one micropit is in cell-cell communication with a cell disposed at the opening of the vertical slot.

### REFERENCE TO PENDING PRIOR PATENT APPLICATIONS

This patent application claims benefit of: (i) pending prior U.S. Provisional Patent Application Ser. No. 62/155,762, filed May 1, 2015 by The Methodist Hospital and Joon Hee Jang et al. for HIGH-RESOLUTION IMAGING OF CELL-CELL COMMUNICATION (Attorney's Docket No. METHODIST-21 PROV); and (ii) pending prior U.S. Provisional Patent Application Ser. No. 62/243,257, filed Oct. 19, 2015 by The Methodist Hospital and Lidong Qin et al. for HIGH-RESOLUTION IMAGING OF CELL-CELL COMMUNICATION (Attorney's Docket No. METHODIST-21R PROV).

The two (2) above-identified patent applications are hereby incorporated herein by reference.

### FIELD OF THE INVENTION

This invention relates to cell imaging in general, and more particularly to high resolution imaging of cell-cell communication.

### BACKGROUND OF THE INVENTION

The ability of cells to communicate with each other is one of the most essential and fundamental activities in eukaryotic organisms. The cells of the immune system communicate with each other through a variety of strategies. One of the most important of these is the formation of an interface structure known as the immunological synapse (IS). Since its original description, an enormous body of evidence has been accumulated showing that the IS plays a pivotal role in the immune response, mediating such vital functions as immune recognition, adhesion, activation and inhibition.

Given the critical role of the IS in immune responses against cancer and infection, it is essential to understand the molecular mechanisms underlying IS formation, signaling and function in real cell-cell conjugates. Conventional confocal microscopy of both fixed and live cells represents the most common imaging technique available to study the IS. Traditionally, immune cells are mixed with target cells or antigen-presenting cells (APCs). After fixation, cell-cell conjugates are imaged under a confocal microscope. However, current methods of imaging the synapse face several limitations. One of the most obvious of these is the low resolution of IS images in lateral cell-cell conjugates (i.e., where the two cells are disposed in side-by-side relation). In this situation the cell pairs extend parallel to the plane of focus of the confocal microscope, and the synaptic interface lies perpendicular to the plane of focus, along the Z axis (FIG. 1A, left). As a result, sequential Z-stacked images need to be taken in order to capture the entire synapse interface. However, because conventional confocal microscopes typically have poor resolution along the Z axis, it is challenging to obtain high-resolution synapse images (FIG. 1A). This is because in this geometry, the resolution at the synapse is greatly reduced due to the elongated point spread function (PSF) of the incident laser beam (FIG. 1A, right). The resolution of the image can be significantly improved by simply rotating the cell pair, positioning the IS on a horizontal imaging plane (FIG. 1B). Recently, to achieve an ideal focal plane for IS imaging, researchers have manually oriented the cells on top of one another using optical tweezers. This technique was able to successfully enhance the resolution with which the synapse could be visualized. However, manually orienting the cells one on top of another with optical tweezers is labor-intensive and expensive.

Furthermore, successful operation of optical tweezers requires extensive training. In addition, even when cell stacking is successfully achieved using optical tweezers, lateral movement of the cells (particularly live cells) can occur during cell imaging, which can disrupt imaging of the synapse interface.

Recently, a micropit system has been developed to achieve high-resolution IS imaging between effector and target cells at low cost. However, the loading efficiency of cells in the micropit system alone is low—about 10-15%. In addition, the frequency of vertical orientation stacking between effector and target cells is also relatively low, which makes imaging time-consuming.

Therefore, development of a novel system for vertically stacking cells would fill a significant unmet need to facilitate the study of IS formation and cell-cell communication.

### SUMMARY OF THE INVENTION

The present invention comprises the provision and use of a new system that allows for high-resolution imaging of the IS with conventional confocal microscopy in a high-throughput manner. By combining micropits and single cell trap arrays, a new microfluidic platform has been developed that allows visualization of the IS in vertically “stacked” cells. This vertical cell pairing (VCP) system has been used to investigate the dynamics of the inhibitory synapse mediated by an inhibitory receptor, programmed death protein-1 (PD-1) and the cytotoxic synapse at the single cell level. In addition to the technique innovation, novel biological findings were demonstrated using this VCP system, including novel distribution of F-actin and cytolytic granules at the IS, PD-1 microclusters in the human natural killer (NK) IS, and kinetics of cytotoxicity. The high-throughput, cost-effective, easy-to-use VCP system, along with conventional imaging techniques, can be used to address a number of significant biological questions in a variety of disciplines.

In one preferred form of the invention, there is provided a vertical cell pairing (VCP) system comprising:

a substrate having a top surface;

at least one micropit formed in the top surface of the substrate, the at least one micropit being sized to seat a cell; and

at least one microtrap positioned adjacent the top surface of the substrate, the at least one microtrap comprising a body having a vertical slot, wherein the vertical slot has an opening and an exit, the vertical slot being sized to pass fluid therethrough but to prevent a cell from passing therethrough;

wherein the at least one microtrap is disposed relative to the at least one micropit so that a cell seated in the at least one micropit is in cell-cell communication with a cell disposed at the opening of the vertical slot.

In another preferred form of the invention, there is provided a method for vertically pairing cells, the method comprising:

providing a vertical cell pairing (VCP) system comprising:
a substrate having a top surface;
at least one micropit formed in the top surface of the substrate, the at least one micropit being sized to seat a cell; and
at least one microtrap positioned adjacent to the top surface of the substrate, the at least one microtrap comprising a body having a vertical slot, wherein the vertical slot has an opening and an exit, the vertical slot being sized to pass fluid therethrough but to prevent a cell from passing therethrough;
wherein the at least one microtrap is disposed relative to the at least one micropit so that a cell seated in the at least one micropit is in cell-cell communication with a cell disposed at the opening of the vertical slot;

flowing a first slurry of cells over the top surface of the substrate and seating a first cell in the at least one micropit; and

flowing a second slurry of cells over the top surface of the substrate so that a second cell is disposed at the opening of the vertical slot in the at least one microtrap.

In another preferred form of the invention, there is provided a vertical cell pairing (VCP) system comprising:

a substrate having a top surface; and

at least one microtrap positioned adjacent the top surface of the substrate, the at least one microtrap comprising a body having a vertical groove and a vertical slot, wherein the vertical groove has an opening and an exit, the vertical groove being sized to seat a pair of cells therein when the pair of cells are vertically aligned, wherein the vertical slot has an opening and an exit, the vertical slot being sized to pass fluid therethrough but to prevent a cell from passing therethrough, wherein the exit of the vertical groove is in fluid communication with the opening of the vertical slot.

In another preferred form of the invention, there is provided a method for vertically pairing cells, the method comprising:

providing a vertical cell pairing (VCP) system comprising:
a substrate having a top surface; and
at least one microtrap positioned adjacent the top surface of the substrate, the at least one microtrap comprising a body having a vertical groove and a vertical slot, wherein the vertical groove has an opening and an exit, the vertical groove being sized to seat a pair of cells therein when the pair of cells are vertically aligned, wherein the vertical slot has an opening and an exit, the vertical slot being sized to pass fluid therethrough but to prevent a cell from passing therethrough, wherein the exit of the vertical groove is in fluid communication with the opening of the vertical slot;

flowing a first slurry of cells over the top surface of the substrate and seating a first cell in the vertical groove of the at least one microtrap; and

flowing a second slurry of cells over the top surface of the substrate and seating a second cell in the vertical groove of the at least one microtrap.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings.

FIG. 1: Optical geometry and VCP system design. (A) The optical geometry (left) for imaging the IS by conventional methods and corresponding simulated point-spread-function (PSF, right) of the excitation beam. (B) The optical geometry (left) for imaging the IS by the VCP system and corresponding simulated PSF (right) of the excitation beam. (C) Overall design of the microfluidic platform and flow pathways during cell loading. VCP ver.3 (see below, including FIG. 18) is shown. (D) Wide field fluorescent microscopic image merged with bright field image of the microfluidic VCP system after cell loading. Red and green channels correspond to K562 and KHYG-1 cells, respectively. Scale bar indicates 100 μm (left) and 20 μm (right), respectively. (E) Percentage of trap-captured cells in each step during the cell loading procedure. The graph shows average and standard deviation (SD) of cell capture efficiency over four different area of the VCP system. The results are from three independent experiments.

FIG. 2: Simulated flow velocity distribution on the top layer. (A) Overview of the flow velocity in VCP ver.3. (B) Flow velocity distribution around a single microstructure without cell. Red lines show bottom layer and white blocks indicate top polydimethylsiloxane (PDMS) structure. (C) Flow velocity distribution changes around a single microstructure with a trapped cell. The flow velocity is pseudo-colored with cool and warm colors indicating low and high flow velocity, respectively.

FIG. 3: Comparison of IS structure by conventional (Conv.) and VCP systems imaged by confocal fluorescence microscopy. CD16-KHYG-1 (NK) cell conjugated with K562 (target) cell on cover glass coated with poly-L-lysine (A) and vertical stack of NK-target cell pair by VCP ver.2 (see below, including FIGS. 16 and 17) (B) are fixed, permeabilized, and stained for F-actin (red), perforin (green), and α-tubulin (cyan). Scale bars indicate 5 μm. The three-dimensional (3D) fluorescent images centered at the IS are z-projected. Magnified areas (8×8 μm, white boxes) from the fluorescent microscope image obtained by using coverslips coated with poly-L-lysine (C) and by using the VCP ver.2 system. (D) Perforin (green) and F-actin (red) are visualized. Scale bars indicate 2 μm. (E) Fluorescence intensity profile of F-actin and perforin was measured across the white line. Fluorescence intensity of the perforin and F-actin is shown in green and red line, respectively. (F) Full width at half maximum (FWHM) and standard error of fluorescence intensity for individual perforin granule along the vertical line (blue line in C and D). (G) Pearson's correlation coefficient (r) was calculated for perforin and F-actin from 3D colocalization analysis. Each dot represents each pair (n=46 for conventional, n=45 for VCP imaging). (H) Pearson's correlation coefficient (r) was calculated from 2D colocalization analysis. The same set of data as (G) was used. (I) Costes P-value (indicating the reliability of Pearson's correlation coefficient analysis) obtained during 2D colocalization analysis. VCP ver.3 was used for (G-I).

FIG. 4: Time-lapse images of “Dispersed→Centralized→Dispersed” (D→C→D) PD-1/PD-L1 clusters by 3D confocal fluorescence microscope using VCP ver.3. (A) shows two examples of a subpopulation of NK-target cell pairs (68.2%, 15 out of 22 observations) whose PD-1/PD-L1 microclusters coalesced, then dispersed. Scale bars indicate 5 μm. (B) Proposed model of PD-1/PD-L1 cluster movement during cell-cell communication within the D→C→D subpopulation.

FIG. 5: Time-lapse images of “stay-Dispersed” (sD) PD-1/PD-L1 clusters by 3D confocal fluorescence microscope. (A) and (B) represent two examples of a subpopulation of PD-1/PD-L1 cluster movement on two cell pairs imaged using VCP ver.3. During imaging, the clusters did not coalesce in this sub-population (22.7%, 5 out of 22 observations). Scale bars indicate 5 μm.

FIG. 6: Time-lapse images of “Dispersed→stay-Centralized” (D→sC) PD-1/PD-L1 clusters (9.1%, 2 out of 22 observations) by 3D confocal fluorescence microscope. (A) Schematic model (left) and time series (right). Fluorescent images of selected time points from live cell imaging by using the VCP ver.2 system for IS formation between PD-L1-mCherry+K562 (bottom, red) and PD-1-GFP+CD16-KHYG-1 (top, green) cells. PD-1-GFP, PD-L1-mCherry, bright field, merged, and colocalization of PD-1 and PD-L1 are presented. Scale bar, 10 μm. (B) Track path of the clusters in the colocalized fluorescence image. Black dashed line indicates central cluster region. (C) Fluorescent image merged with bright field image after disconnecting negative pressure.

FIG. 7: Kinetics of cytotoxicity mediated by NK cells imaged under wide-field fluorescence microscope. (A) Live cell time-lapse images from the VCP ver.3 system used to establish kinetics of CD16-KHYG-1 (red)-mediated cytotoxicity against K562 (green) target cell. Scale bars indicate 100 μm (left) and 10 μm (right), respectively. (B) Mean fluorescent intensity (MFI) of K562 cells paired with CD16-KHYG-1 cells (gray), K562 cells alone (green), and CD16-KHYG-1 cells alone (red) during the 6-hour acquisition. (C-F) Classification of killing kinetics of NK cells when paired with K562 target cells. (C) “Slow decay” of normalized MFI from K562 cells conjugated with NK cells for 6 hrs. (D) “Single-drop” of normalized MFI from K562 cells conjugated with NK cells (left) and quantification of “single-drop” occurrence in K562 cells after conjugation with NK cells (right). (E) “Fast decay” of normalized MFI from K562 cells conjugated with NK cells for 6 hrs. (F) “Multiple-drop” of normalized MFI from K562 cells conjugated with NK cells (left) and quantification of “multiple-drop”occurrence in K562 cells after conjugation with NK cells (right). Black lines indicate average of the each population.

FIG. 8: Previous and current version of vertical cell pairing systems which have been developed. Different parameters of the vertical cell pairing systems are compared side-by-side.

FIG. 9: The step-by-step VCP system fabrication procedure and cell loading efficiency. (A) A representative photo of vertical cell pairing system and photolithograpy procedure. A detailed procedure is described in the Materials And Methods section below. (B) Selected image of KHYG-1 (green) and K562 (red) cells trapped in the VCP ver.3 system with different cell loading density. (C) Loading efficiency of the VCP system at different cell loading density. The plot represents data from three independent tests. Error bars indicate standard deviation.

FIG. 10: Fabrication and dimension of the two PDMS layers of VCP ver.3. (A) The overall photomask design of the bottom layer. (B) The overall photomask design of the top layer. Scale bar indicates 5 mm. (C) The bottom PDMS layer containing the micropit array. (D) The top PDMS layer containing the single cell trap array. (E) The aligned microfluidic VCP system are imaged via bright field microscopy.

FIG. 11: Comparison of IS structure by conventional (Conv.) and VCP systems imaged by confocal fluorescence microscopy. Vertical stack of CD16-KHYG-1 NK cell-K562 target cell pair using VCP ver.3 (A) and CD16-KHYG-1 NK-K562 target cell pair on cover glass coated with poly-L-lysine (B) and are fixed, permeabilized, and stained for F-actin (red), perforin (green). Scale bars indicate 5 μm for zoom out and 2 μm for zoom in.

FIG. 12: Verification of the cell lines by flow cytometry. (A) Histogram of PD-1-GFP⁺ CD16-KHGY-1 cells (left) and cells stained with PD-1-Alexa Fluor647 antibody (right). The unstained CD16-KHYG-1 cells served as negative control (Gray). (B) Histogram of PD-L1-mCherry⁺ K562 cells (left) and cells stained with PD-L1-FITC antibody (right). The unstained K562 cells served as negative control (Gray).

FIG. 13: Schematic view of a prior art micropit system for vertically pairing cells.

FIG. 14: Schematic view of a microtrap for vertically pairing cells.

FIG. 14A: Schematic front view of the microtrap shown in FIG. 14.

FIG. 15: Schematic view of a novel vertical cell pairing (VCP) system (VCP ver.1—see below, including FIG. 15) for vertically pairing cells.

FIG. 15A: Schematic front view of the vertical cell pairing (VCP) system (VCP ver.1) shown in FIG. 15.

FIG. 16: Schematic view of a novel vertical cell pairing (VCP) system (VCP ver.2) for vertically pairing cells.

FIG. 16A: Schematic front view of the vertical cell pairing (VCP) system (VCP ver.2) shown in FIG. 16.

FIG. 17: Schematic view of the vertical cell pairing (VCP) system (VCP ver.2) shown in FIG. 16, but with a first cell type and a second cell type positioned in the VCP system.

FIG. 18: Schematic view of a novel vertical cell pairing (VCP) system (VCP ver.3) for vertically pairing cells.

FIG. 18A: Schematic front view of the vertical cell pairing (VCP) system (VCP ver.3) shown in FIG. 18.

### DETAILED DESCRIPTION OF THE PREFERRED EMBODIMENTS

The present invention comprises the provision and use of a novel system to study the IS horizontally in a high-resolution and high-throughput manner. Combining micropits with single cell trap arrays, this newly-developed microfluidic platform provides various technical novelties that answer the previously-discussed difficulties in IS imaging. The vertical cell orientation of the paired cells enables the IS to be imaged in a horizontal plane and enhances resolution on both fixed and live cell imaging. This vertical cell pairing (VCP) system can minimize the lateral cell drift at the focal plane and constrain the conjugated cells in the vertical position. This system can also capture more than 3000 conjugates at a time with high loading efficiency. Using this VCP system, a “face-to-face” look at the structure of the human natural killer (NK) cell IS is presented. Compared to conventional cell-cell conjugates, the organization of F-actin at the IS can be clearly observed at the VCP system with high-resolution using conventional confocal microscopy. The VCP system of the present invention was able to detect the positioning of perforin-positive lytic granules over regions of low F-actin density at the IS, a detail which has been previously reported under super-resolution microscopy, but one that has proved difficult to image using conventional approaches. In addition, bright F-actin puncta that were segregated from the cytolytic granules were observed at the center of NK synapse, which is usually indiscernible with conventional confocal microscopy. Furthermore, the novel dynamics of PD-1 microclusters at NK synapse and target cell lysis mediated by NK cells at a real cell-cell interface were also observed in the VCP system. Thus, the VCP system provides a high-throughput, high-efficiency, and user-friendly approach to IS imaging that not only successfully addresses many of the problems of previous techniques, but also possesses broad potential applications in a variety of other biological disciplines.

### Novel Vertical Cell Pairing (VCP) System

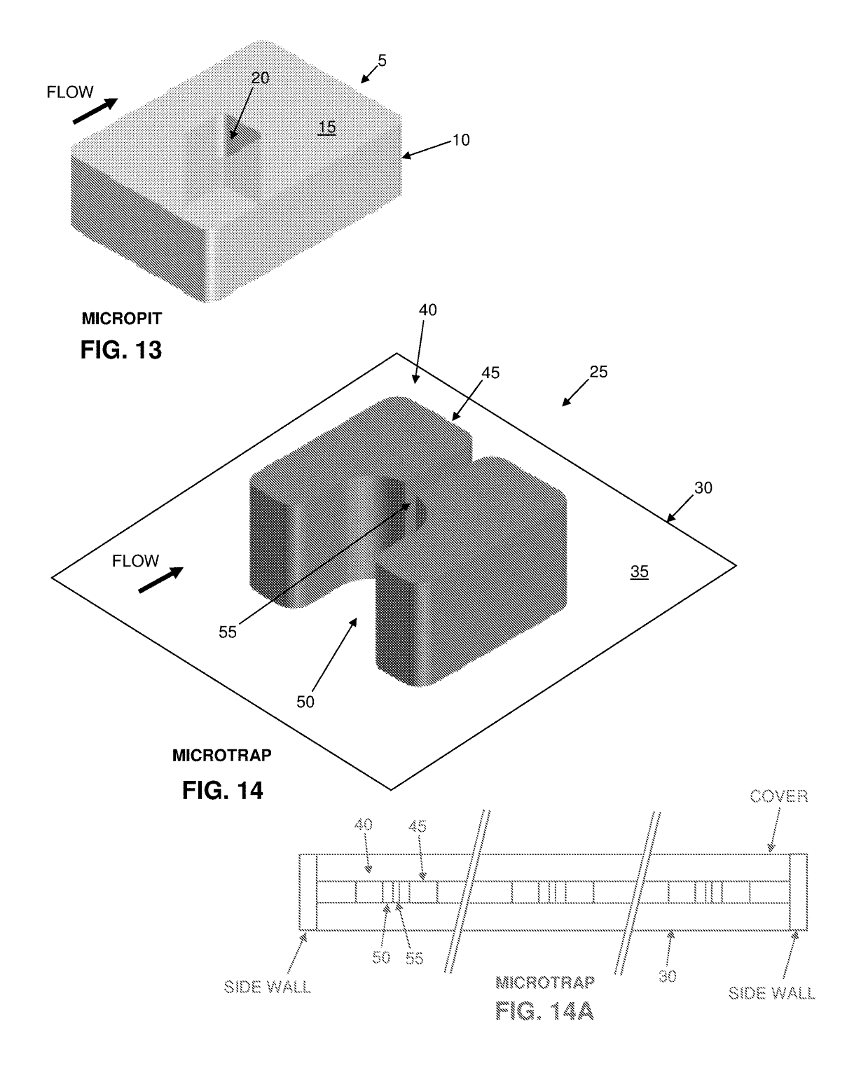

Looking first at FIG. 13, there is shown a prior art micropit system 5, such as the micropit system developed by Biggs et al. Micropit system 5 is characterized by a substrate 10 having a top planar surface 15, and a micropit 20 formed therein. Micropit 20 is sized so that when a first slurry of cells of a first cell type is flowed over top planar surface 15, a cell of that first cell type can find its way into micropit 20. Micropit 20 is further sized so that when a second slurry of cells of a second cell type is thereafter flowed over top planar surface 15, a cell of that second type can also find its way into micropit 20, so that the cell of the second cell type is seated atop the cell of the first cell type. It will be appreciated that with the prior art micropit system 5 shown in FIG. 13, a cover (not shown) is disposed atop, and spaced from, substrate 10 so as to create a channel through which the first and second cell slurries can be successively passed.

Prior art micropit system 5 suffers from a number of disadvantages, including (i) low yield (due to the difficulty of inducing a cell to enter micropit 20), and (ii) poor cell seating (the cell of the second cell type may move out of vertical alignment with the cell of the first cell type even when both cells are seated in micropit 20).

FIGS. 14 and 14A show a microtrap system 25 formed in accordance with the present invention. Microtrap system 25 is characterized by a substrate 30 having a top planar surface 35, and a microtrap 40 disposed on top planar surface 35. Microtrap 40 comprises a body 45 characterized by a vertical groove 50 and a vertical slot 55. Vertical groove 50 is sized so that when a first slurry of cells of a first cell type is flowed over top planar surface 35, a cell of that first cell type can find its way into vertical groove 50. Vertical groove 50 is further sized so that when a second slurry of cells of a second cell type is thereafter flowed over top planar surface 35, a cell of that second cell type can also find its way into vertical groove 50, so that the cell of the second cell type is seated atop the cell of the first cell type. It will be appreciated that in a preferred construction, a cover (not shown) is disposed atop, and spaced from, substrate 30 so as to create a channel through which the first and second cell slurries can be successively passed.

Microtrap system 25 suffers from a number of disadvantages, including (i) low yield (due to the difficulty of inducing a cell to enter vertical groove 50), and (ii) low pairing efficiency for two different types of cells (i.e., the same types of cells are paired in majority of the microtraps).

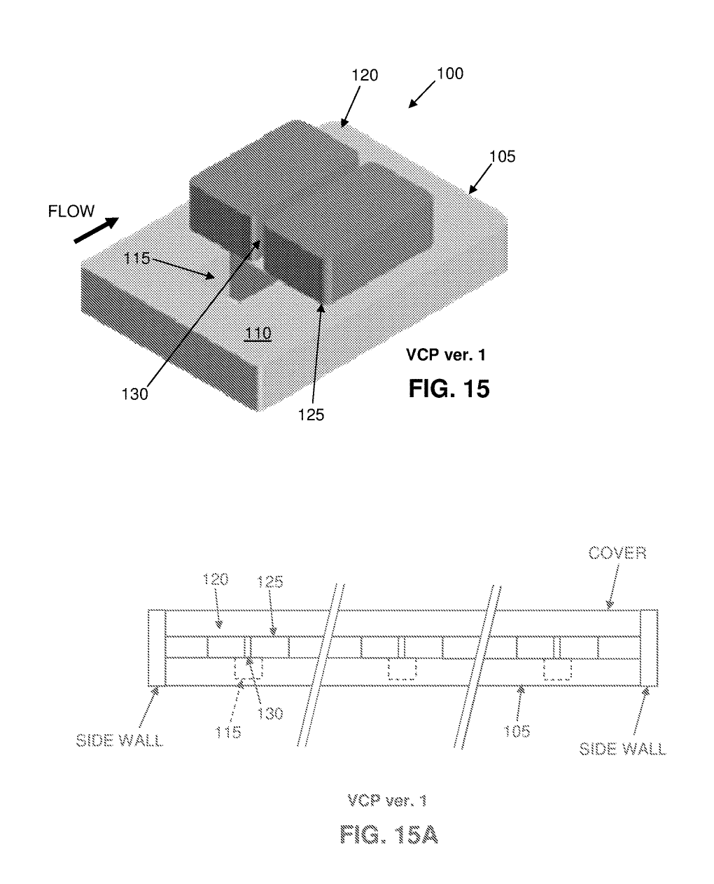

In accordance with the present invention, and looking now at FIGS. 15 and 15A, there is provided a vertical cell pairing (VCP) system 100 (VCP ver.1). Vertical cell pairing system 100 is characterized by a substrate 105 having a top planar surface 110, and a micropit 115 formed therein. Micropit 115 is sized so that it can receive a cell of a first cell type, as will hereinafter be discussed. Vertical cell pairing system 100 further comprises a microtrap 120 (sometimes hereinafter referred to as a “micropillar”) disposed on top planar surface 110. Microtrap 120 comprises a body 125 characterized by a vertical slot 130. Vertical slot 130 is sized so that when a slurry of cells is flowed over top planar surface 110, a cell can find its way to the mouth of vertical slot 130. Microtrap 120 is disposed on top planar surface 110 such that vertical slot 130 is longitudinally aligned with micropit 115.

On account of the foregoing, when a first slurry of cells of a first cell type is flowed over top planar surface 110, a cell of the first cell type will be captured by vertical slot 130 of microtrap 120 directly over micropit 115. Thereafter, when cell pairing system 100 is subjected to a centrifugal force (e.g., using a centrifuge), the cell of the first cell type captured by microtrap 120 will be caused to enter, and seat in, micropit 115. A washing step can then be effected (e.g., flowing a buffer solution over top planar surface 110) so as to wash away any cells which have not entered micropit 115. Then a second slurry of cells of a second cell type is flowed over top planar surface 110 so that a cell of the second cell type is captured by microtrap 120, i.e., so that the cell of the second cell type is disposed atop, and in communication with, the cell of the first cell type which is captured in micropit 115. If desired, cell pairing system 100 may be subjected to a further application of centrifugal force (e.g., using a centrifuge) so as to ensure that the cell of the second cell type (captured by microtrap 120) is disposed in secure contact with the cell of the first cell type (captured in micropit 115). In a preferred form of the invention, a cover (not shown in FIG. 15) is disposed atop, and spaced from, substrate 105 so as to create a channel through which the first and second cell slurries can be successively passed.

Significantly, by pairing microtrap 120 with micropit 115, vertical cell pairing system 100 provides (i) high yield (due to the ease with which a cell of the first cell type can be captured by microtrap 120 and then loaded into micropit 115, and due to the ease with which a cell of the second cell type can be captured by microtrap 120 adjacent to the cell of the first cell type), and (ii) the ensured alignment of the cell of the second type with the cell of the first cell type (due to the alignment of the vertical slot 130 of microtrap 120 with micropit 115).

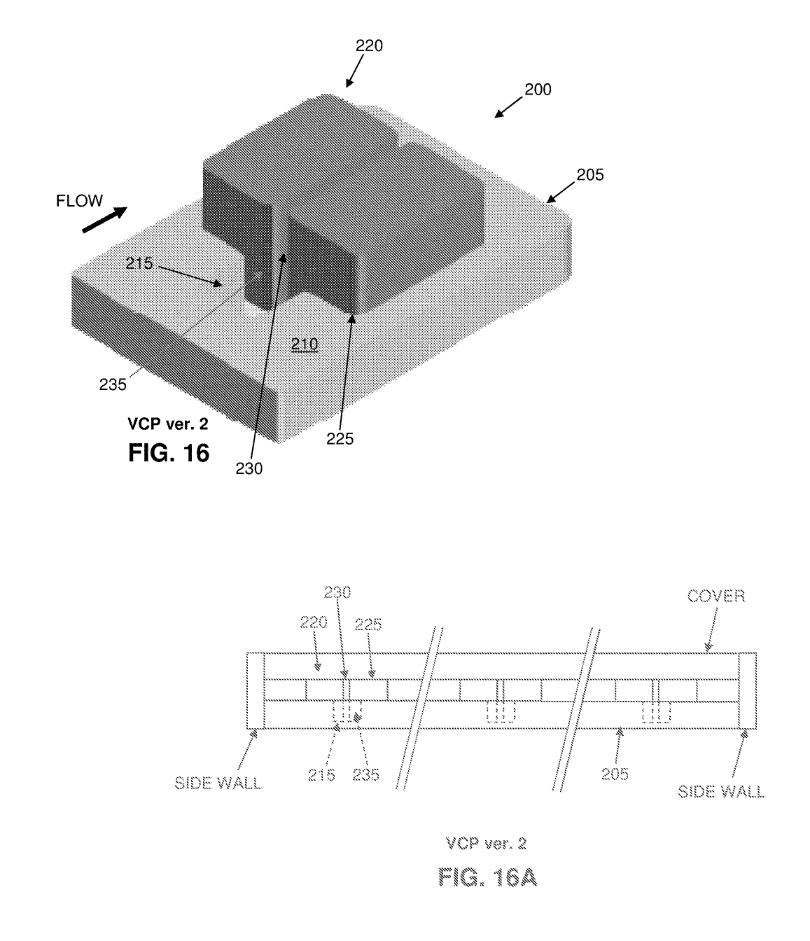

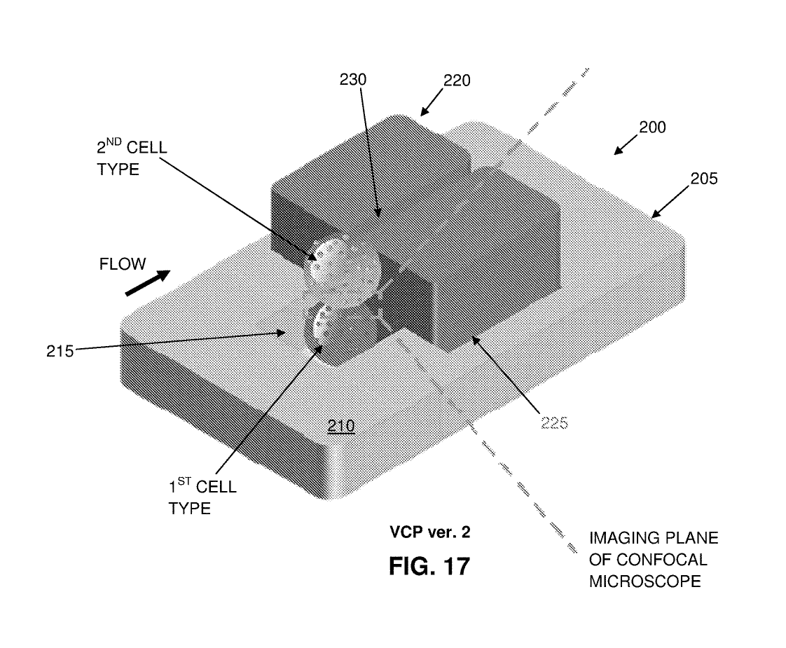

FIGS. 16, 16A and 17 shown another vertical cell pairing system 200 (VCP ver.2) formed in accordance with the present invention. Vertical cell pairing system 200 is substantially the same as vertical cell pairing system 100 discussed above, except that (i) micropit 215 of vertical cell pairing system 200 is elongated in the direction of flow, and (ii) body 225 of microtrap 220 includes a tongue 235 sized to seat in a portion of micropit 215, whereby to ensure that vertical slot 230 of microtrap 220 is always aligned with the longitudinal centerline of micropit 215. It should be appreciated that, by forming tongue 235 on body 225 of microtrap 220, it is possible to provide vertical cell pairing system 200 on two slides which “snap” together to form the complete assembly: the first slide comprises body 225 of microtrap 220 (or a plurality of bodies 225 of a plurality of microtraps 220), with tongue 235 (or a plurality of tongues 235) extending away from the slide, and the second slide comprises substrate 205 and micropit 215 (or a plurality of micropits 215) arranged to receive tongue 235 of body 225. Thus, the alignment of tongue 235 of microtrap 220 with micropit 215 can assist in the assembly of vertical cell pairing system 200.

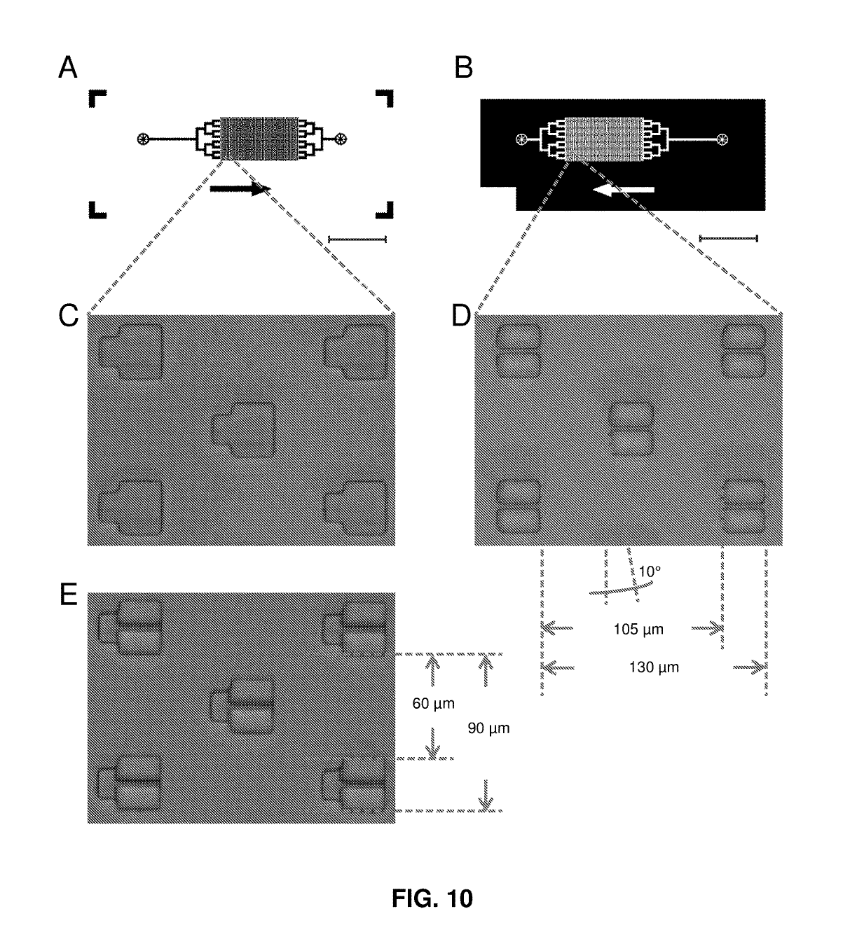

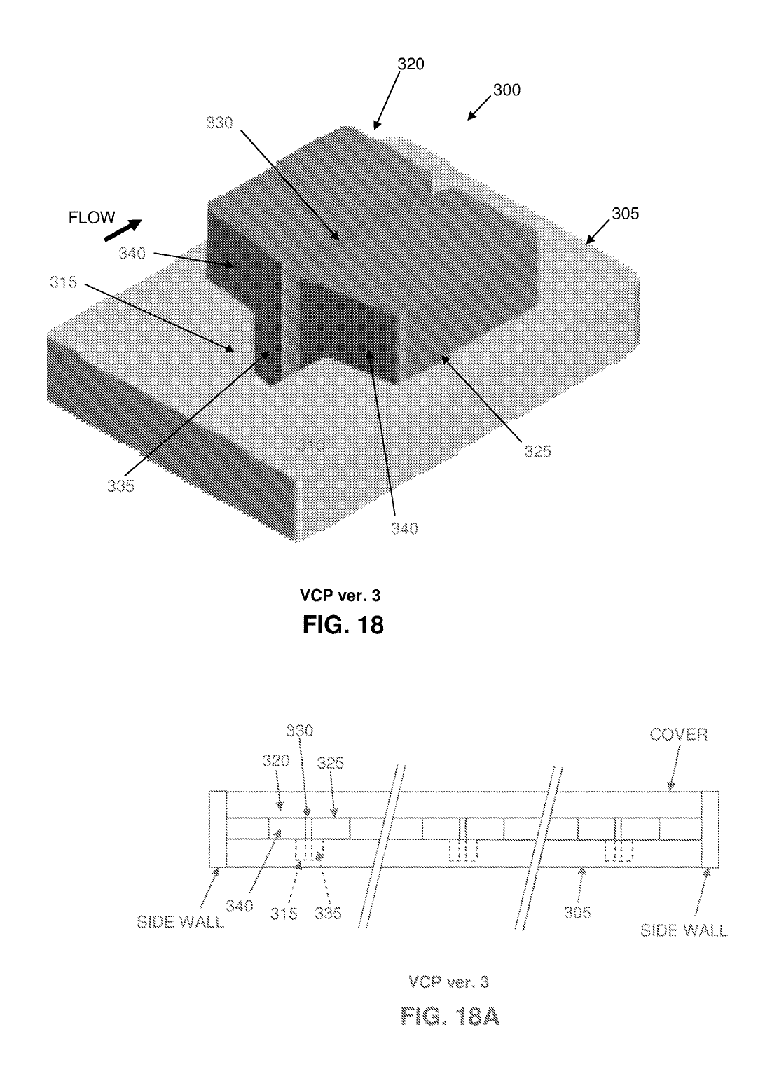

FIGS. 18 and 18A show another vertical cell pairing system 300 (VCP ver.3) formed in accordance with the present invention. Vertical cell pairing system 300 is substantially the same as vertical cell pairing system 200 discussed above, except that the “upstream” surfaces 340 of microtrap 320 that define the mouth of vertical slot 330 are “swept back” slightly from the geometry shown in in FIGS. 16 and 17, preferably at an angle of approximately 5 to approximately 35 degrees, e.g., at the 10 degree angle shown in FIG. 10. This “swept back” configuration is highly advantageous, since it ensures that only cells directly aligned with vertical slot 130 of microtrap 320 will be retained on the upstream face of microtrap 320. More particularly, this “swept back” configuration ensures that only one cell will be caught at the mouth of vertical slot 330, and ensures that the caught cell will be in direct vertical alignment with micropit 315. In this way, “swept back” configuration of surfaces 340 of microtrap 320 can also increase the loading efficiency of vertical cell paring system 300.

### Materials and Methods

1. Microfluidic VCP System Fabrication

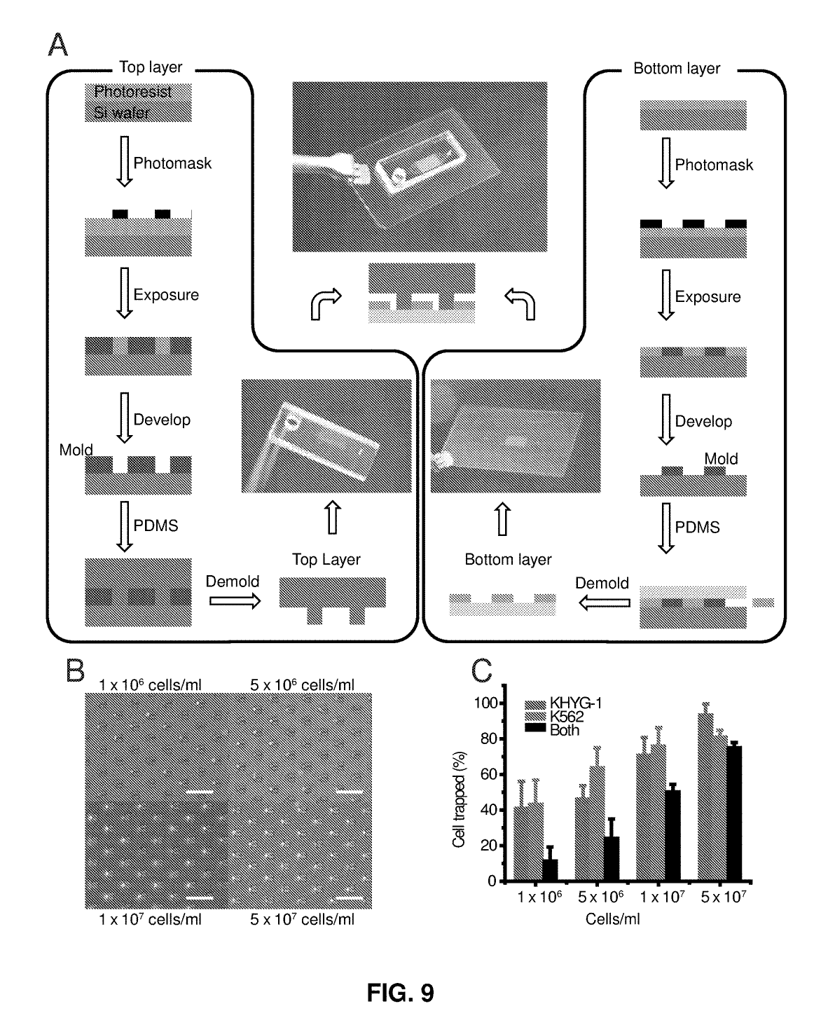

The schematic flow of the fabrication procedure is summarized in FIG. 9. Negative photoresist (SU-8 3025, MicroChem, Newton, Mass.) was coated on an oxygen plasma-cleaned silicon(100) wafer (500 μm thickness, Silicon Quest International, USA). The spin coater (WS-400BZ, Laurell technology Co., USA) was set at 500 rpm for 10 s to evenly spread the photoresist on the wafer. The photoresist was spun at 2900 rpm for 30 s, yielding a 30 μm thickness for the top mold, and at 4000 rpm for 60 s, yielding a 15 μm thickness for the bottom mold. The photoresist was soft baked for 3 min at 95° C. and exposed to UV light through the photo mask. After baking the photoresist at 65° C. for 1 min and 95° C. for 4 min and developing, the micropit and trap array molds were obtained. The mold was coated with trimethylchlorosilane (TMCS) at atmospheric pressure for 30 min.

Polydimethylsiloxane (PDMS, Sylgard 184, Dow Corning Corp.) was mixed with curing agent at a ratio of 10:1. The micropit array mold (bottom mold) was dipped into the PDMS and pressed on a number 1.5 cover glass (170 μm thickness) with 40 Newtons by a heated mechanical press (CH4386, Carver press). The PDMS was cured at 80° C. for 2 hours, and the mold was removed to obtain the bottom layer. The PDMS was poured onto the trap array mold (top mold) and cured at 77° C. for 1 hr. The PDMS was lifted from the mold to obtain the top layer. The inlet hole (4 mm diameter) and negative pressure port (0.5 mm diameter) were punched (Accu-Punch, Syneo) into the top PDMS layer. A drop of methanol was applied for lubrication and the top layer was snapped on the bottom layer to assemble the two layers.

2. Cells Culture

The K562 myelogenous leukemia cell line (American Type Culture Collection, ATCC) was cultured in RPMI medium (Gibco, USA). The KHYG-1 human NK cell line (provided by Dr. David T. Evans, Harvard Medical School) was maintained in R10 medium consisting of RPMI 1640 medium (Gibco, USA) supplemented with 10% fetal bovine serum (FBS, Gibco, USA), 10 mM HEPES (Gibco, USA), 1 mM sodium pyruvate (Cellgro, USA), 2 mM L-Glutamin (Gibco, USA), and 1% MEM non-essential amino acids solution (Gibco, USA). Additionally, 1 μg/ml cyclosporine A (CsA, Sigma-Aldrich, USA), 50 μg/ml Primocin, and 10 U/ml interleukin-2 (IL-2) were freshly added into the R10 medium for each passage. The culture medium for CD16-KHYG-1 cells was replaced with CsA-free medium one day before use. The Human Embryonic Kidney 293T (HEK293T) cells were cultured in DMEM (Gibco, USA) with 10% FBS, 10 mM HEPES, and 2 mM L-Glutamine.

3. Plasmids and Transduction of CD16-KHYG-1 and K562 Cell Lines

To generate the PD-1-GFP construct, a full length of PD-1-GFP fusion protein sequence (OriGene, MD) was amplified by the primers 5′-AATCCGGAATTCGCCGCCGCGATCGCCATGC-3′ (Forward) and 5′-AATCGCGGATCCTTAAACTCTTTCTTCACC-3′ (Reverse). The PCR product digested with EcoRI and BamHI (Thermo Scientific) was ligated with EcoRI and BamHI digested lentivector pCDH (23). Similarly, to generate the C-terminal mCherry-tagged PD-L1 construct, the PD-L1 cDNA (OriGene, MD) was amplified by the primers 5′-AATCCGGAATTCATGAGGATATTTGCTGTCT-3′ (Forward) and 5′-AATCGCGGATCCCGTCTCCTCCAAATGTGTA-3′ (Reverse). The PCR product was digested with EcoRI and BamHI and ligated with an mCherry-N1 vector (Clontech) to generate C-terminal mCherry-tagged PD-L1. The sequence of PD-L1-mCherry was then amplified by the primers 5′-TAGAGCTAGCGAATTATGAGGATATTTGCTGTCTTTA-3′ (Forward) and 5′-ATTTAAATTCGAATTTCACGCCTTGTACAGCTCGTCC-3′ (Reverse). The PCR product was inserted into EcoRI digested pCDH lentivector by the In-fusion cloning system (Clontech). All plasmids were verified by sequencing.

To generate the PD-1-GFP⁺ CD16-KHYG-1 and PD-L1-mCherry⁺ K652 cell lines, both CD16-KHYG-1 and K562 cell lines were transduced with the pCDH cDNA cloning and expression lentivirus system (SBI, System Biosciences), respectively. The lentivirus was generated by co-transfecting Human Embryonic Kidney 293T (HEK293T) cells with a lentiviral vector containing PD-1-GFP or PD-L1-mCherry and three packaging plasmids (pMLg/pRRE, pRSV-Rev, pMD2.g) by Lipofectamine reagent (Invitrogen). Briefly, 0.8 μg total DNA (0.128 μg pCDH, 0.32 μg pMLg/pRRE, 0.16 μg pRSV-Rev, 0.192 μg pMD2.g) was mixed with 4 μl Lipofectamine and added into one well of 293T cells cultured in a 6-well plate with approximately 90% confluence. Viral particles were harvested and filtered by a 0.45 μm filter (GE) after 48 hours transfection. Then, 2×10⁵ CD16-KHYG-1 or K562 cells suspended in 4 ml R10 medium were infected with 4 ml viral supernatant. Transduced cells were cultured for 24 hours in the presence of 8 μg/ml polybrene and subsequently sorted by an Aria II cell sorter (BD). The expression of PD-1-GFP and PD-L1-mCherry were verified by flow cytometry (BD LSR Fortessa).

4. Cell Loading

The fabricated VCP microfluidic system was placed on a vacuum desiccator for 15 min. A 1 ml syringe was connected to the port through a tube to generate negative pressure and a drop of 2% bovine serum albumin (BSA) in phosphate buffered saline (PBS) was introduced into the inlet. The VCP system was incubated for 30 min at 37° C. Then R10 medium was subsequently used to replace the BSA solution in the VCP system. 10 μl of the first cell suspension with a concentration range of 10⁶-10⁷ cells/ml was added into the inlet. The flow rate of 15 μl/min was used to seed the cells using a syringe pump. After 30 s of seeding, the rest of the cell suspension was washed with PBS three times. The microfluidic VCP system was disconnected from the syringe and centrifuged (Sorvall Legend X1R, Thermo scientific) at 2000 rpm (700×g) for 10 min to spin down the cells into micropit array. After centrifugation, the syringe was re-connected and the second cell suspension was added into the inlet. The parameters for seeding the second cell suspension were the same as those for the first cell suspension.

For live cell imaging, the K562 cells stably expressing PD-L1-mCherry were first injected into the VCP system. The VCP system was mounted on the inverted fluorescence microscope stage. A single cell was focused upon with a 63x oil immersion objective lens. Then the CD16-KHYG-1 cells expressing PD-1-GFP were injected. After the effector cells were anchored on top of the target cells, the cell suspension in the inlet was replaced with fresh cell medium containing 25 mM HEPES buffer. Live cell imaging was performed at the synapse plane at every 30 s for 10 min. During the live cell imaging, the flow rate is maintained at 0.5 μl/min. For fixed cell imaging, the CD16-KHYG-1 cells were injected first. The VCP system was incubated for 1 hour at 37° C. The cell-cell conjugates were fixed by 4% formaldehyde solution in PBS for 15 min, then washed with PBS for 5 min. Permeabilization buffer containing 5% of normal donkey serum (NDS) and 0.5% Triton X-100 in PBS was added into the VCP system. The VCP system was incubated in 4° C. overnight. The permeabilization buffer was washed with PBS for 5 min. Then, primary antibody against α-tubulin (Abcam) in antibody buffer containing 3% NDS and 0.5% Triton X-100 in PBS was pumped into the VCP system. The VCP system was incubated at 4° C. overnight. The antibody buffer was washed with PBS for 5 min. The cells were stained by fluorescently labeled secondary antibody (Life Technologies). The antibody buffer was washed with PBS for 5 min, and finally, a drop of ProLong Gold antifade reagent mounting medium (Life Technologies) was added into the VCP system.

5. Confocal Microscope

High-resolution images were captured with a confocal fluorescence microscope (Leica TCS SP8, Leica Microsystem) equipped with a 63x oil immersion objective lens (NA 1.47, Leica Microsystem). In the fixed cell images, z-stacks of tubulin, perforin and actin were sequentially captured. The high-resolution live cell images were carried out at 37° C. 4.5 μm z-stacks centered at the IS were acquired to account for movement within the live cell. The fluorescence from PD-1-GFP, PD-L1-mCherry, and the bright field image was detected simultaneously. The images were acquired by LAS AF software (Leica) and analyzed with ImageJ and Imaris (Bitplane) software.

### Results

1. Design Optimization of Vertical Cell Pairing Microfluidic System

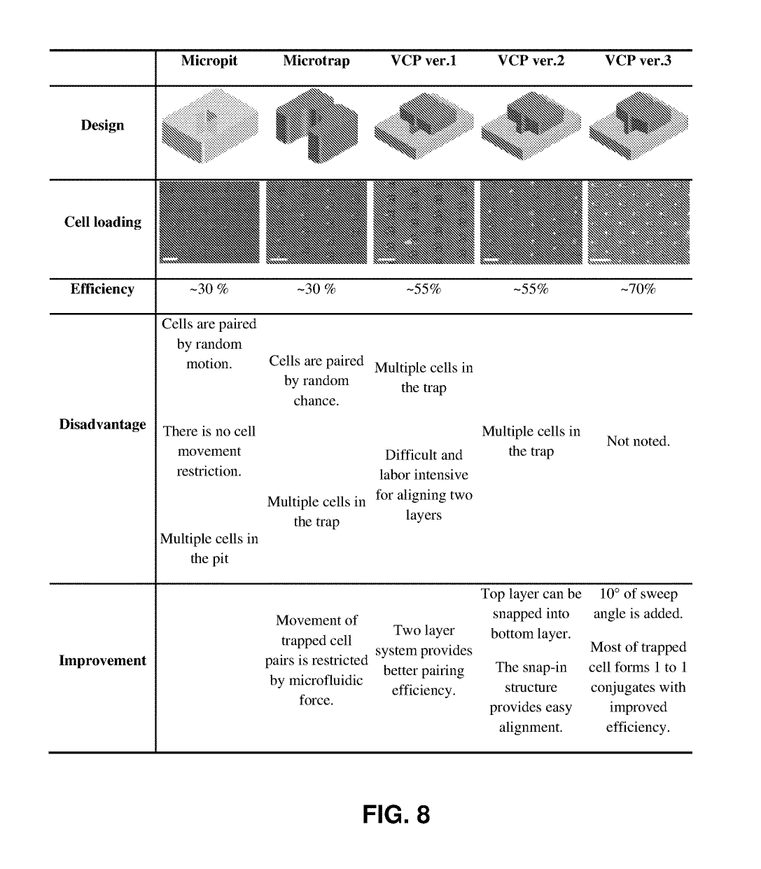

The development and optimization process of the VCP system design is summarized in FIG. 8. A single layer micropit and microtrap design was previously tested, from which ˜30% of heterogeneous cell pair was achievable with optimized loading conditions (FIG. 8). By combining the micropit with the micropillar (microtrap), the pairing efficiency increased to ˜55% (VCP ver.1). However, aligning the two layers in microscale is time-consuming and labor intensive. Furthermore, it is nearly impossible to make perfect alignment over the whole area due to PDMS shrinkage during the curing process. Thus, the VCP system was designed with a snap-in structure (VCP ver.2). The two layers were aligned by snapping the trap structures of the top layer together with the through-holes of the bottom layer by one-click alignment, where the micropits of the bottom layer line up with the microtraps of the top layer (FIG. 9). This method can achieve precise alignment of all microstructures without the need for any specialized tools. This method also prevents alignment shift problems and PDMS shrinkage ratio mismatches that occur in the previous two layer design. The 15 μm height of the micropit array is overlaid with the 30 μm height of the microtrap array, creating a 15 μm high flow channel between the two PDMS layers. Further optimization of the design was made by adding a sweep angle to the micropillar (microtrap) (VCP ver.3). The sweep angle is preferably approximately 5 to approximately 35 degrees. In one preferred form of the invention, the sweep angle is approximately 10 degrees. This 10 degree sweep angle on the micropillar (microtrap) provides better one-on-one cell pairs with improved loading efficiency of more than 70%. Detailed dimensions of the VCP Ver. 3 system are shown in FIG. 10.

The fabrication method is described in greater detail in the Materials And Methods section above. Briefly, a polydimethylsiloxane (PDMS) pre-polymer-coated mold of the bottom layer, which contains 4000 individual micropits, was directly stamped onto a No. 1.5, 170 μm thick cover glass. The top layer, which includes the microtrap array, was prepared by standard photolithography procedures.

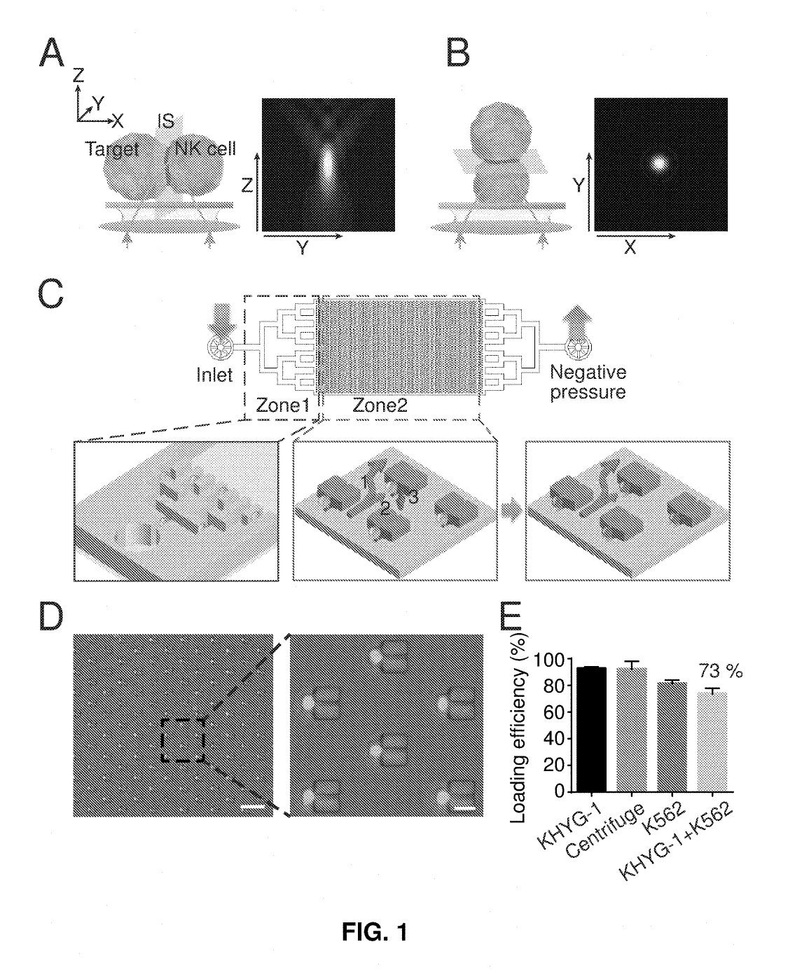

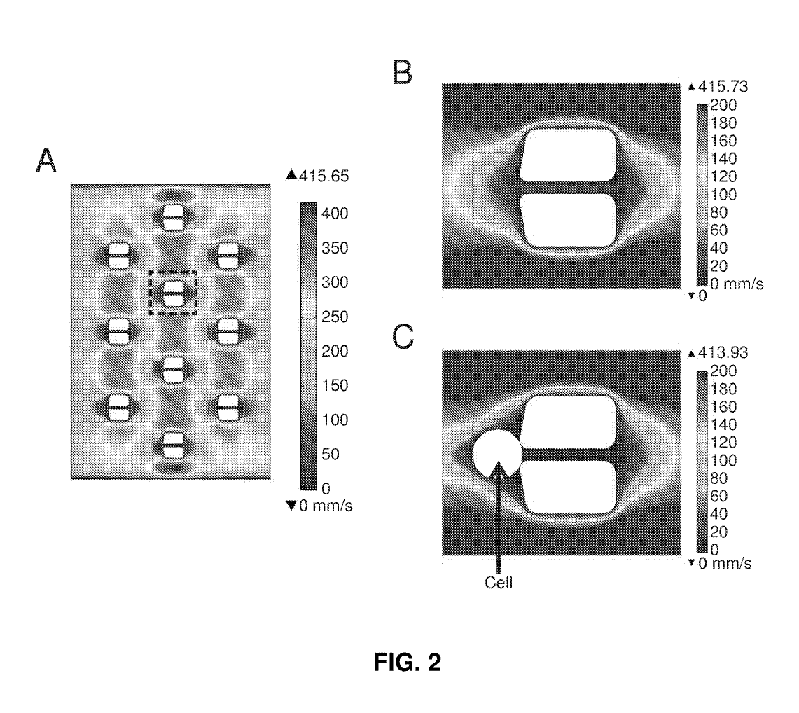

The microfluidic VCP system features four areas including inlet, negative pressure port, zone 1, and zone 2 to control the flow rate and direction (FIG. 1C). Media and cell suspensions were injected via the inlet. Flow pressure is generated by a 1 ml syringe connected to the negative pressure port. Zone 1 consists of microchannels to break up the cell clusters and evenly distribute the flow of medium and suspended cells into Zone 2. Zone 2 contains the microtrap array, which captures the cells. There are two pathways for governing the cell loading mechanism (FIG. 1C). The horizontal pathway (labeled as pathway 1 and 2, respectively) flows around the microtrap structure and passes through a 3 μm gap between traps. Once the cell suspension is injected via the inlet, the cell preferentially takes pathway 1 due to the high flow rate. When multiple cells are taking the same pathway, the flow is disturbed, and a single cell can be anchored in between the trap by taking pathway 2. Once a cell is wedged into the 3 μm gap between the trap, the flow distribution around the trap is changed due to the blockage by the trapped cell. Thus, the subsequent cells take pathway 1, leaving a single cell trapped in the microstructure, which constrains lateral cell movement. Detailed flow velocity distributions are simulated in FIG. 2. The low-flow velocity area in FIG. 2B is extended after trapping a cell between the micropillars (microtraps), which contributes to reduce flow resistance (FIG. 2C). Thus, subsequent cells preferentially bypass the micropillars (microtraps). Of note, the previous study shows that the cavity under the laminar flow does not affect overall flow characteristics, while the laminar flow might introduce vortex in the cavity. Therefore, the microtrap structures were omitted to demonstrate the flow distribution.

The gravitational force (red arrow in FIG. 1C) pulling the cell down into the micropit is negligible in this system. The micropit is initially filled with cell suspension medium. The approximate density of the medium is 1.0 g/ml, according to the manufacturer, and that of the blood cell is 1.1 g/ml. Thus, the horizontal flow pinning the cell against the microtrap easily overwhelms the gravitational force acting on the cell. However, artificially increasing the gravitational force by centrifugation readily brings the cell down into the micropit. After this, the second cell suspension was injected and anchored on top of the first cells by the same mechanism (FIGS. 1C and D).

To test the loading efficiency of the VCP system, the fraction of the captured cells in each step was measured as shown in FIG. 1. First, an NK cell line, KHYG-1, expressing CD16 (CD16-KHYG-1; green in FIG. 1D) was injected into the VCP system with 92.8±1.1% trapping efficiency. The percentage of the captured cells was maintained at 92.2±5.9% after centrifugation. The sequential injection of target K562 (a human immortalized myelogenous leukemia line) cells (red in FIG. 1D), achieved a capture efficiency of 81.3±2.7%. Finally, the percentage of the microstructures trapping both KHYG-1 and K562 cells was 73.7±4.4% (FIG. 1E). The factors that affect loading efficiency (such as flow rate and cell loading density) were independently assessed. For the flow rate, 15 μl/min for cell loading and 0.5 μl/min for live cell imaging were used to minimize shear stress on cells. The loading efficiency increased as a function of cell loading density (FIGS. 9B and 9C). Throughout the experiment, ˜10⁶ cells suspended in 50 it medium were used and it was possible to image the cell pairs with 60-70% efficiency. These results demonstrate that it is possible to successfully fabricate a VCP system that is capable of co-capturing vertically “stacked” target and effector cells in a high-throughput, high-efficiency manner.

2. High-Resolution Imaging Intracellular Structure of IS on Fixed Cells by VCP System

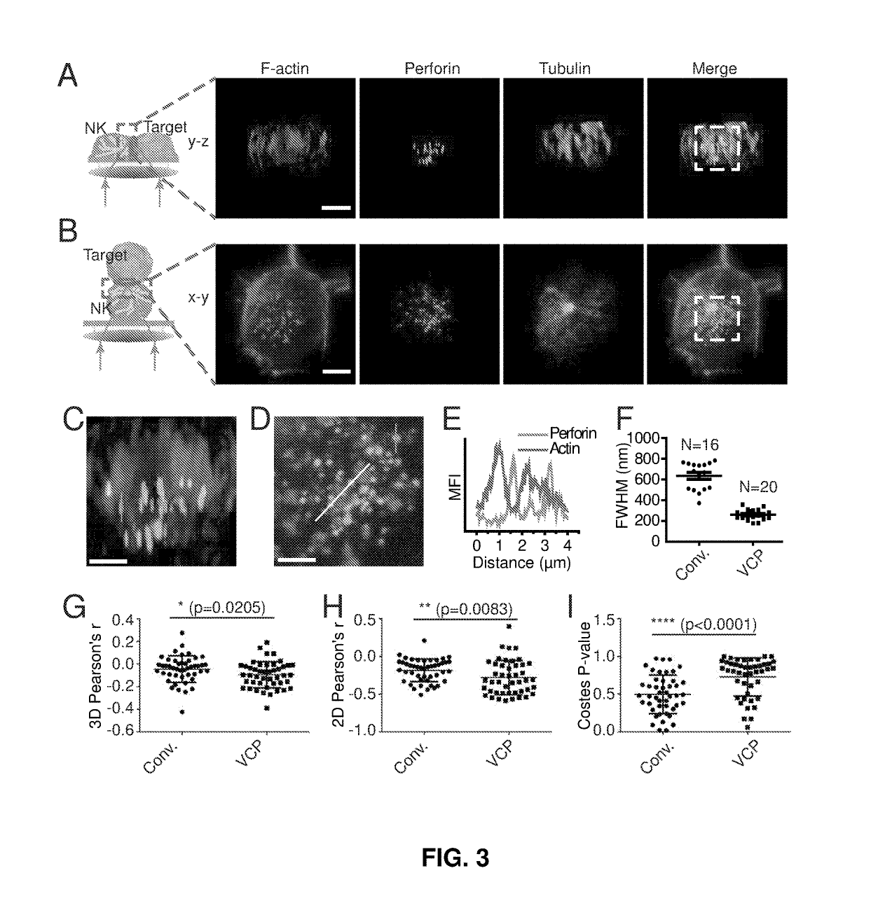

After successfully developing this VCP system, further testing was done to determine whether it would permit high-resolution imaging with a conventional confocal microscope, a common instrument in most research institutes. CD16-KHYG-1 cells were used as effector cells and the K562 cell line as susceptible target cells. The CD16-KHYG-1 effector cells were loaded into the VCP system, followed by the target cells. After fixation, cell-cell conjugates were stained for F-actin, perforin, and tubulin. As a control, conventional microscopy slides were prepared simultaneously, as previously described. Three-dimensional (3D) images were acquired in each fluorescence channel. After reconstruction of the 3D-stack, a z-projection of the interface between two cells was presented (FIG. 3).

The representative image obtained by the VCP system demonstrates significant improvements over that of the image obtained by the conventional method in two respects. First, the present system prevents deformation of the IS. In the traditional method, cells spread out due to the interaction between the plasma membrane and the polylysine-coated coverslip (FIG. 3A). In contrast, the VCP system maintained the IS in its natural shape without distortion (FIG. 3B). Second, the VCP system provides superior spatial resolution compared to the conventional method. A zoomed-in area taken from the 3D confocal images acquired by both the conventional coverslips (FIG. 3C) and VCP system (FIG. 3D) was compared. The round-shape of perforin-positive cytolytic granules was easily distinguished in the image measured by the VCP system, whereas the cytolytic granules appeared elongated in the image achieved by the conventional method. Furthermore, the detail of the fine F-actin meshwork in the image obtained by the VCP system (FIG. 3D) was greater than that of the image acquired by the conventional approach. Perforin-positive cytolytic granules were observed positioned over low-density areas of F-actin (FIG. 3E), in agreement with previous observations. To quantify the improvement in resolution, the full-width-at-half-maximum (FWHM, an indicator of the granule size) of the fluorescence line intensity profiles of the cytolytic granules was measured. The FWHM of the cytolytic granules measured with the VCP system was significantly smaller than that of those measured using the conventional approach (FIG. 3E).

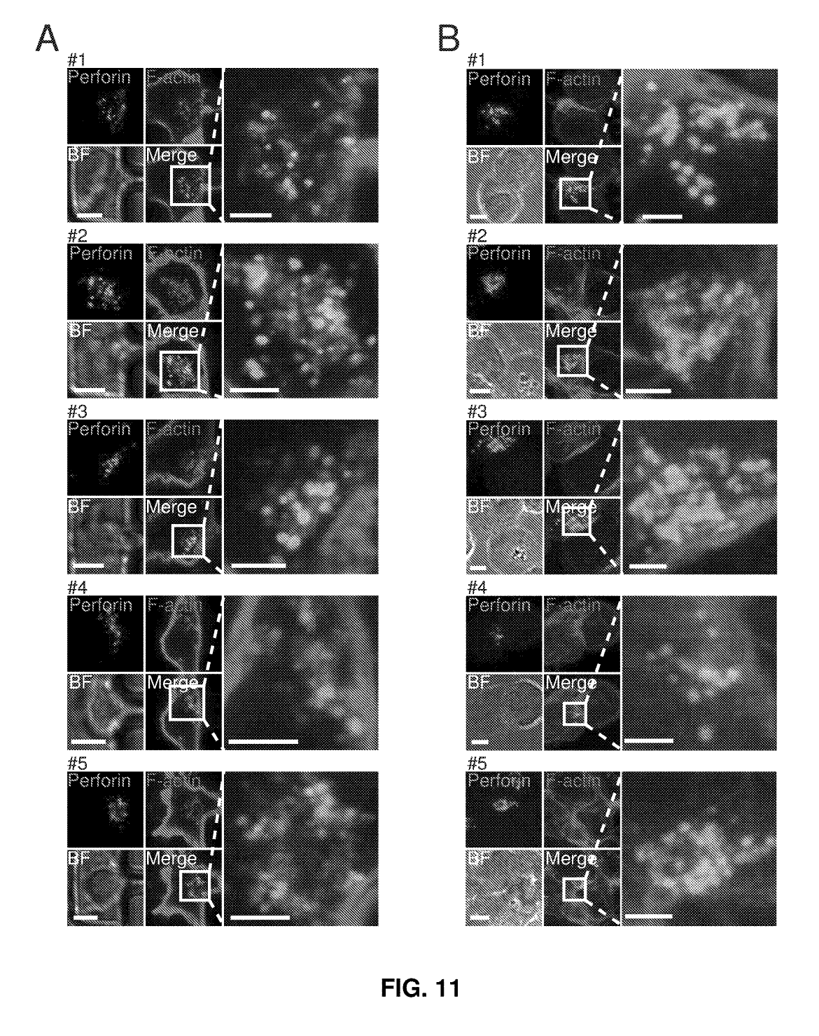

To further quantify the unique features of F-actin puncta that appear well segregated from the perforin positive cytolytic granules in the IS, colocalization analysis was carried out and Pearson's correlation coefficient (Pearson's r) was calculated for the images obtained using the VCP system and the conventional method (FIG. 3 and FIG. 11). F-actin formed numerous puncta at the center of IS, where the perforin positive cytolytic granules were well segregated by these F-actin puncta (a phenomenon that could not be appreciated in the side view using the conventional method), which is distinct from the previous observations that F-actin is cleared at the center of the synapse. To further quantify these observations, the Pearson's r from 3D and 2D colocalization analysis was calculated by Imaris (Bitplane) and ImageJ software, respectively (FIGS. 3G and H). A striking negative correlation between perforin and F-actin at the NK cell synapse has been observed by using both conventional and now VCP imaging methods. To quantitatively determine which method provides a more reliable set of analysis, the Costes P-value was extracted from 2D colocalization analysis (FIG. 31). While the conventional method shows an average of 50% confidence level, most of the p-value obtained by the VCP system shows confidence levels close 100%. Together, these results demonstrate that the resolution with which the IS can be visualized in the VCP system is superior to that of conventional methods.

3. High-Resolution Imaging of Dynamics of PD-1 Microclusters on Live Cells by VCP System

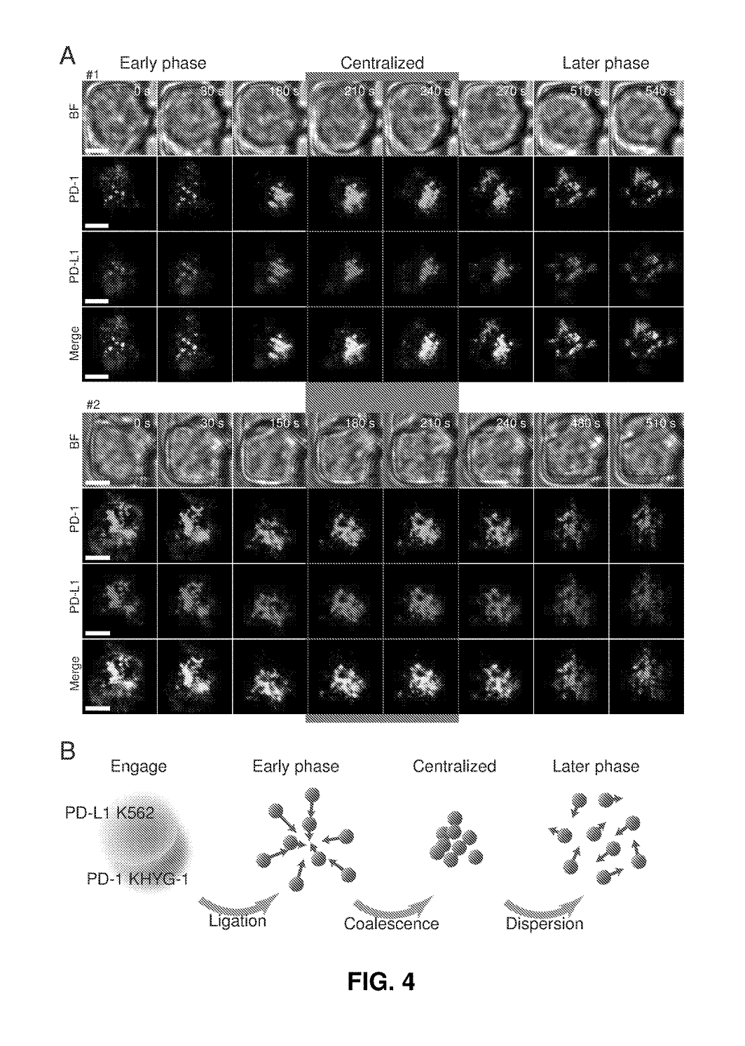

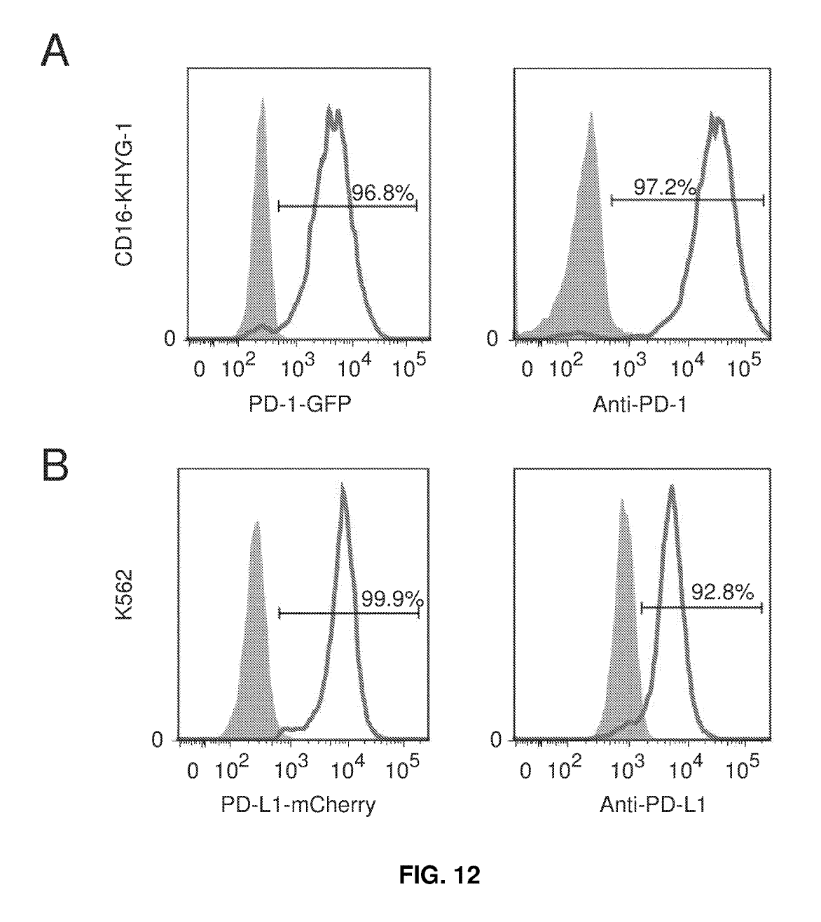

Live imaging provides unprecedented information on the dynamics of the inhibitory IS, which cannot be obtained from images of fixed cells. After successfully demonstrating the high-resolution imaging of the IS made possible by the VCP system, the VCP system was further tested to determine whether the VCP system allows live-cell imaging of the IS. Here, a fluorescently tagged programmed cell death-1 (PD-1) was used, an important inhibitory receptor expressed on lymphocytes, as a mode to study the dynamics of the inhibitory synapse. To test whether the VCP system could be used to visualize the dynamics of PD-1 at the IS, CD16-KHYG-1 cells expressing PD-1-GFP were used, together with susceptible K562 target cells expressing PD-1 ligand (PD-L1)-mCherry (FIG. 12). Live cell imaging revealed that PD-1 and PD-L1 microclusters formed three distinct patterns at the NK IS. To further quantify the three different patterns, 22 live cell pairs were imaged independently (FIGS. 4-6). To capture the early phase of IS formation, PD-1-GFP⁺ CD-16 KHYG-1 cells were loaded into the VCP system first. After mounting the VCP system on the microscope, the PD-L1-mCherry⁺ K652 target cells were added into the VCP system. The 22 cell pairs were categorized into three different patterns based on the movement of PD-1 microcusters at the NK IS. The majority of conjugates (68.2%, 15 out of 22) showed that the PD-1/PD-L1 clusters coalesce in the center of the IS at the early phase (<5 min after interaction) and then disperse into the periphery of the IS in the later phase (FIG. 4). This dynamic PD-1/PD-L1 movement in the synapse may be termed as “Dispersed→Centralized→Dispersed” (abbreviated as “D→C→D”), as shown in FIG. 4. Given that this pattern was observed in the majority of all conjugates (68.2%), a model of the dynamic movement of PD-1/PD-L1 clusters is proposed (FIG. 4B) in which PD-1/PD-L1 microclusters coalesced to the center of IS in the early phase of IS formation (within 1-2 min). Then, these temporally centralized clusters dissociated from each other, and dispersed within the entire synapse with random movement.

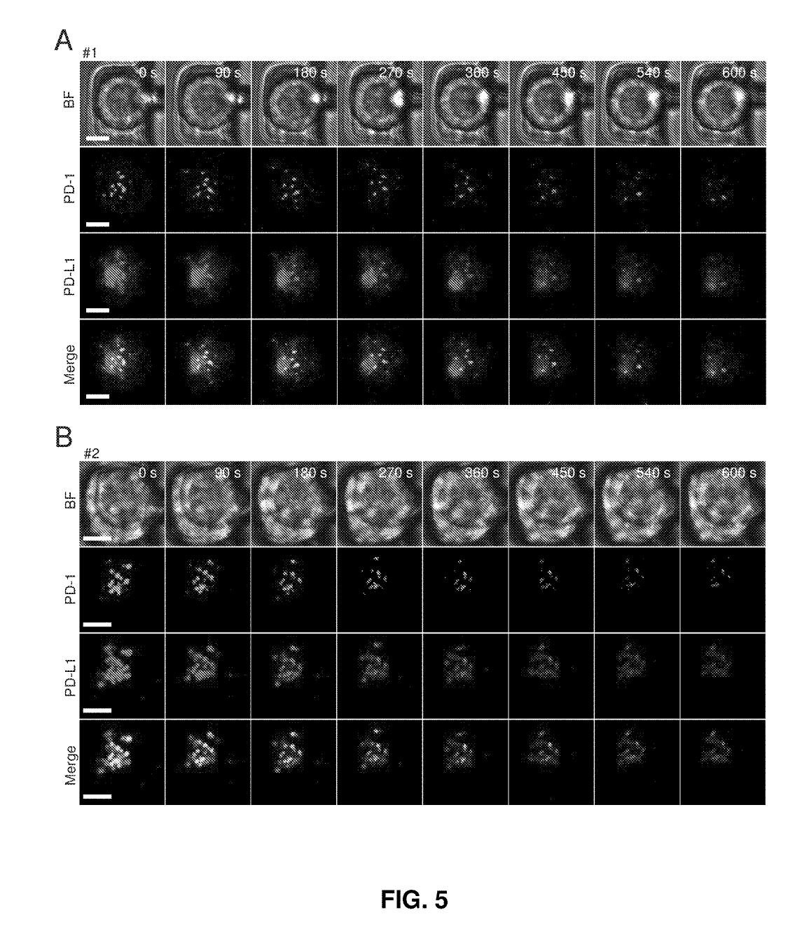

There was also observed a second pattern of PD-1/PD-L1 microcluster movement within the synapse, in which PD-1/PD-L1 does not form a centralized cluster during the 10 min time-lapsed imaging acquisition (FIG. 5). These PD-1/PD-L1 microclusters (22.7%, 5 out of 22 conjugates) remain dispersed during the acquisition (FIG. 5). This pattern may be referred to as “stay dispersed” (abbreviated as “sD”), as shown in FIG. 5.

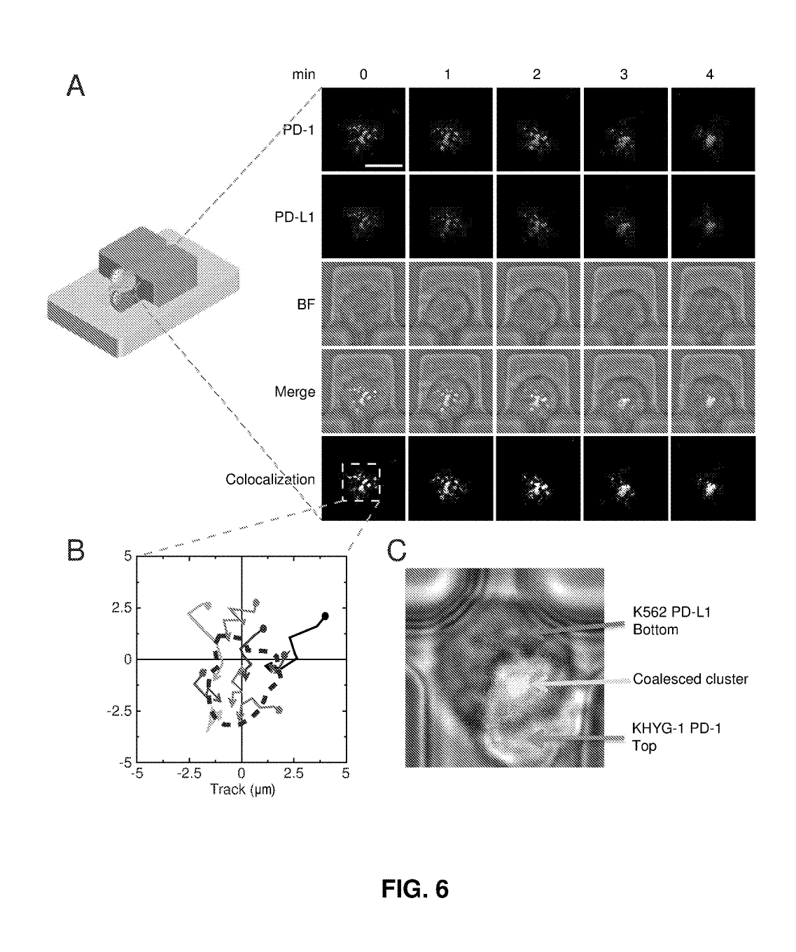

In addition to the “D→C→D” and “sD” patterns, there was also observed that a small minority of PD-1/PD-L1 microclusters (9.1%, 2 out of 22 conjugates) form at the periphery of the IS and coalesce at the center of the synapse, where they remain clustered (FIG. 6A). This dynamic of PD-1/PD-L1 movement in the synapse may be referred to as “Dispersed→Centralized” (abbreviated as “D→C”). Consistent with previous observations, PD-1 microclusters were similar to HLA-E clusters that formed between SLBs and human primary NK cells. To quantify the temporal dynamics of PD-1 and PD-L1 microclusters, the trajectory of individual microclusters was tracked over time. As shown in FIG. 6B, 9 microclusters accumulated rapidly at the center of synapse. The averaged velocity of each trajectory was calculated (19±5 nm/s, n=9). To confirm stable inhibitory synapse formation on vertically orientated NK and K562 cells in the VCP system, medium was continuously flowed during imaging, with a constant flow rate of 0.5 μl/min. Once the flow was removed, the CD16-KHYG-1 cell being imaged (green, top) was released from the top layer of the single cell trap, but remained anchored to the top of the K562 cell (red, bottom; FIG. 6C).

From these observations it was concluded that the dynamics of inhibitory synapse microclusters in live cells can be readily observed in the VCP system. It is believed that this is the first observation of single microcluster dynamics at IS in a real cell-cell conjugate with high resolution. Meanwhile, the distinct patterns of PD-1/PD-L1 microclusters in the IS is a striking feature of inhibitory synapses formed between PD-1 positive NK cells and PD-L1 positive target cells.

4. Kinetics of Live NK Cell Cytotoxicity Detected by the VCP System

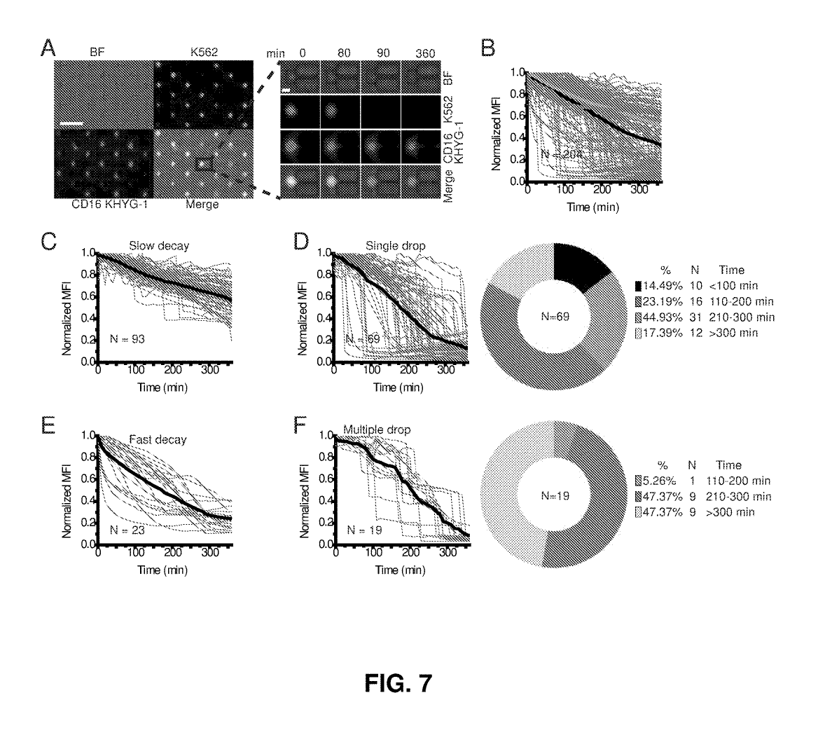

Next, the VCP system was tested to see whether cytolytic killing could be monitored by the VCP system in live cells. To test this, K562 cells were loaded with calcein AM green viability dye. To distinguish the effectors from the target cells, CD16-KHYG-1 cells were labeled with CellTracker red. Cell-cell conjugates were imaged by wide-field fluorescence microscopy (FIG. 7). The disappearance of calcein AM green fluorescence signal was used as a readout for effective killing of target cells. A clear decrease in the green fluorescent signal was observed in the K562 cells co-cultured with CD16-KHYG-1 effector cells (FIG. 7A). A relatively stable red signal and a sudden decrease in green signal were observed in one cell-cell conjugate. To quantify the kinetics of cytotoxicity in the VCP system, 204 effector-target cell pairs were analyzed. The fluorescence intensity from K562 cells labeled with calcein AM green were measured every 10 min (FIG. 7B). The kinetics of cytotoxicity elicited by NK cells was categorized by the fluorescence intensity profile of K562 cells. Here, the complete disappearance of calcein AM was used as a readout for cell death. Around 45.6% of K562 cells (93 out of 204 conjugates) still fluoresced after 6-hour conjugation with NK cells, suggesting that these K562 cells remained alive (FIG. 7C). This fluorescence profile may be referred to as “slow decay”. The average fluorescence decay profile of K562 cells was comparable to that of unconjugated K562 cells (green line in FIG. 7B).

In addition to the “slow decay” fluorescence profile from calcein AM green, there was also observed three different kinetics of disappearance of calcein AM green in K562 cells. The second pattern of calcein AM (33.8%, 69 out of 204 conjugates) displayed a “single-drop” profile, indicating lysis of K562 cells by NK cells (FIG. 7D). Consistent with previous observations, the average time for NK-mediated cytotoxicity was about 222.2 minutes (SD=84.3 min) after injection (FIG. 7D).

The third fluorescence profile of calcein AM (11.3%, 23 out of 204 conjugates) was termed “fast-decay” (FIG. 7E). In the “fast-decay” dynamic, the level of calcein AM green eventually became undetectable within 6 hours post-conjugation, suggesting a different cytotoxicity mechanism elicited by NK cells.

Notably, multiple stepwise decreases over time (“multiple-drop” profile) in calcein AM fluorescence (9.3%, 19 out of 204 conjugates) were also observed (FIG. 7F), suggesting a potential mechanism with multiple cytolytic hits from NK cell. Average cell death time in this subpopulation was determined to be 293.2 minutes (SD=49.1 min).

Altogether, these data show that the kinetics of cytotoxicity over time can be readily detected by the VCP system in both a high-throughput manner and at the single cell level, refinements which are all lost by conventional 4-hour ⁵¹Cr release assay, which only provides a blanket measure of killing activity over the entire culture after a certain time period, and is incapable of distinguishing between individual cytolytic and non-cytolytic cell pairings.

### DISCUSSION

The immunological synapse is a critical platform for mediating an effective immune response in both the adaptive and innate immune systems. Current research on synaptic geometry is restricted in artificial systems such as antibody-coated coverslips or glass-supported planar lipid bilayer systems. The present invention provides a novel high-throughput VCP microfluidic system to study the IS in an actual cell-cell communication setting. A detailed protocol for generating this system is provided here. Using this system, a high-resolution image of the IS formed between and effector and target cell has been achieved under conventional confocal microscopy. Additionally, potential applications of this system have been demonstrated by investigating the dynamics of PD-1/PD-L1 microcluster formation at the IS, providing the first observation of microclusters at the IS in vertically oriented real cell-cell conjugates, as well as the kinetics of killing at the single cell level. The present invention provides a user-friendly VCP system and demonstrates the feasibility and application of this novel technique for studying cell-cell communications in a variety of disciplines. In addition, by using this VCP system, it is possible to provide insights on several significant biological questions.

Compared to the micropit system developed by Biggs and colleagues, the VCP system possesses two distinct advantages. The rate of successful vertical conjugates is significantly higher in the VCP system (˜73% vs. 10-15%) due to the novel combination of the micropit system with a single cell trap array, which efficiently guides the cells into the pits. Secondly, the VCP system addresses one of the major difficulties of the original micropit system, wherein there was an unavoidable tradeoff between loading efficiency (i.e., success rate of two-cell pairs entering single pits) and vertical orientation. In the micropit system, a larger pit diameter allows for higher loading efficiency, but because of the large pericellular area of the micropit, the top cell will often not be perfectly aligned on top of the bottom one.

Conversely, a narrower pit diameter significantly decreases the loading efficiency. Guiding the cells into narrow micropits by the single cell trap array, the VCP system is able to maintain a vertical orientation without compromising loading efficiency.

As a result of its innovative design, the VCP system has addressed a number of significant questions. The organization of F-actin at cytotoxic immunological synapse is still highly controversial. In the T cell IS, it has been shown that F-actin is totally cleared from the center of the synapse, which facilities degranulation events at the center of IS. However, this phenomenon is different in the NK cell synapse. Using super-resolution stimulated emission depletion (STED) and structured illumination microscopy (SIM), two independent research groups have shown that F-actin is not totally cleared at the center of IS. Instead, a punctuated, low density F-actin meshwork has been observed at the center of the NK cell synapse, where perforin-positive cytolytic granules are well segregated by the F-actin puncta. In agreement with these observations, perforin-positive cytolytic granules are seen positioned at low-density F-actin regions in the VCP system, a crucial detail that is usually indiscernible with conventional confocal microscopy. Using this VCP system, for the first time, it was cleanly observed that perforin-positive cytolytic granules are located among low density F-actin regions in the center of the IS formed between NK and target cells, without the need for super-resolution imaging techniques. Therefore, the results obtained with the VCP system support the phenomenon that F-actin “hypodensities” are present at the center of the IS in human NK cells and that cytolytic granules converge at these hypodensities.

In addition to the information gained from fixed-cell imaging, live imaging with the VCP system provides additional, critical information on the dynamics of inhibitory immunological synapses not available from previous studies of fixed NK cells. Programmed cell death protein 1 (PD-1) is an emerging immune checkpoint protein highly up-regulated in T, B, and NK cells in the setting of chronic viral infection and tumorigenesis. Similar to other inhibitory receptors such as KIR and CD94/NKG2A, the intracellular domain of PD-1 contains an immunoreceptor tyrosine inhibitory motif (ITIM), which plays a critical role in NK cell inhibition.

Engagement of PD-1 with PD-L1 inhibits cytotoxic killing of K562 target cells by CD16-KHYG1 cells, which indicates the occurrence of an inhibitory synapse. However, whether NK cells can form a stable inhibitory synapse is still controversial. Previous studies have shown that NK cells do not form stable synapses in the presence of inhibitory receptors. Using the VCP platform, it was observed that the microclusters of PD-1 and PD-L1 at the IS coalesce into a central cluster at the synapse. This pattern is reminiscent of previous studies that show that human primary NK cells form stable inhibitory synapse on lipid bilayers carrying HLA-E, a ligand for inhibitory receptor CD94/NKG2A. In addition, the results obtained by this novel VCP system demonstrate three distinct PD-1/PD-L1 microcluster patterns at the NK IS, which is the first to describe the dynamics of PD-1 microclusters in a real cell-cell conjugate at high resolution without the need of an expensive super-resolution imaging system.

An understanding of the forces that govern the stability of the IS is essential to identify its function. One of the most important functions of the IS is to mediate directed cytotoxicity towards target cells. Typical methods of monitoring NK cell cytotoxicity, such as ⁵¹Cr release assays with obvious radiation safety implications, are expensive and time-consuming. The VCP platform discussed herein allows rapid, quantitative, high-throughput functional measurements to monitor NK cell cytotoxicity in real time. The disappearance of the dye in the target cell within a single pit provides a clear readout for cell death at the single cell level, unobscured by the presence of other effector-target pairs, which can be an issue in a large open-well system. Therefore, in addition to providing a platform for enhanced study of the IS, the VCP system provides an alternate method for studying IS-mediated cellular cytotoxicity.

Thus it will be seen that there has been developed a novel, high-throughput VCP system to study cell-cell communications for both fixed and live cell imaging. The feasibility and potential application of this VCP system has been successfully demonstrated. This high-throughput and user-friendly VCP system offers a powerful new imaging platform that can be used to address a number of significant questions in immunology and cell biology, such as single cell analysis, cell fusion, cell-cell communication, and cell surface ligand mobility.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A vertical cell pairing (VCP) system comprising:
a substrate having a top surface;
at least one micropit formed in the top surface of the substrate, the at least one micropit being sized to seat a cell; and
at least one microtrap positioned adjacent the top surface of the substrate, the at least one microtrap comprising a body having a vertical slot, wherein the vertical slot has an opening and an exit, the vertical slot being sized to pass fluid therethrough but to prevent a cell from passing therethrough;
wherein the at least one microtrap is disposed relative to the at least one micropit so that a cell seated in the at least one micropit is in cell-cell communication with a cell disposed at the opening of the vertical slot.

2. A vertical cell pairing (VCP) system according to claim 1 wherein the opening of the vertical slot is substantially aligned with one end of the at least one micropit.

3. A vertical cell pairing (VCP) system according to claim 1 further comprising a cover and a pair of side walls, such that the substrate, the cover and the pair of side walls together define a fluid passageway therebetween.

4. A vertical cell pairing (VCP) system according to claim 3 wherein the at least one microtrap is secured to the substrate.

5. A vertical cell pairing (VCP) system according to claim 3 wherein the at least one microtrap is secured to the cover.

6. A vertical cell pairing (VCP) system according to claim 1 wherein the opening of the vertical slot overlies a portion of the at least one micropit.

7. A vertical cell pairing (VCP) system according to claim 6 wherein the body of the at least one microtrap comprises a tongue sized to be seated within the at least one micropit.

8. A vertical cell pairing (VCP) system according to claim 7 wherein the vertical slot of the at least one microtrap extends into the tongue of the at least one microtrap.

9. A vertical cell pairing (VCP) system according to claim 6 further comprising a cover and a pair of side walls, such that the substrate, the cover and the pair of side walls together define a fluid passageway therebetween.

10. A vertical cell pairing (VCP) system according to claim 9 wherein the at least one microtrap is secured to the substrate.

11. A vertical cell pairing (VCP) system according to claim 9 wherein the at least one microtrap is secured to the cover.

12. A vertical cell pairing (VCP) system according to claim 6 wherein the body of the at least one microtrap comprises a pair of surfaces disposed adjacent to the opening of the vertical slot, and further wherein the pair of surfaces are swept back relative to the opening of the vertical slot.

13. A vertical cell pairing (VCP) system according to claim 12 wherein the pair of surfaces are swept back at an angle of between approximately 5 and approximately 35 degrees relative to the opening of the vertical slot.

14. A vertical cell pairing (VCP) system according to claim 13 wherein the pair of surfaces are swept back at an angle of approximately 10 degrees.

15. A method for vertically pairing cells, the method comprising:
providing a vertical cell pairing (VCP) system comprising:
a substrate having a top surface;
at least one micropit formed in the top surface of the substrate, the at least one micropit being sized to seat a cell; and
at least one microtrap positioned adjacent to the top surface of the substrate, the at least one microtrap comprising a body having a vertical slot, wherein the vertical slot has an opening and an exit, the vertical slot being sized to pass fluid therethrough but to prevent a cell from passing therethrough;
wherein the at least one microtrap is disposed relative to the at least one micropit so that a cell seated in the at least one micropit is in cell-cell communication with a cell disposed at the opening of the vertical slot;
flowing a first slurry of cells over the top surface of the substrate and seating a first cell in the at least one micropit; and
flowing a second slurry of cells over the top surface of the substrate so that a second cell is disposed at the opening of the vertical slot in the at least one microtrap.

16. A method according to claim 15 wherein the first cell is seated in the at least one micropit by flowing the first slurry of cells over the top surface of the substrate so that the first cell is aligned with, or seated in, the at least one micropit, and then centrifuging the vertical cell pairing (VCP) system so as to ensure that the first cell is seated in the at least one micropit.

17. A method according to claim 15 wherein, after the second cell is disposed at the opening of the vertical slot in the at least one microtrap, centrifuging the vertical cell pairing (VCP) system so as to ensure that the second cell is in cell-cell communication with the first cell.

18. A method according to claim 15 wherein the first cell and the second cell are of different cell types.

19. A method according to claim 15 wherein the opening of the vertical slot is substantially aligned with one end of the at least one micropit.

20. A method according to claim 15 wherein the vertical cell pairing (VCP) system further comprises a cover and a pair of side walls, such that the substrate, the cover and the pair of side walls together define a fluid passageway therebetween.

21. A method according to claim 20 wherein the at least one microtrap is secured to the substrate.

22. A method according to claim 20 wherein the at least one microtrap is secured to the cover.

23. A method according to claim 15 wherein the opening of the vertical slot overlies a portion of the at least one micropit.

24. A method according to claim 23 wherein the body of the at least one microtrap comprises a tongue sized to be seated within the at least one micropit.

25. A method according to claim 24 wherein the vertical slot of the at least one microtrap extends into the tongue of the at least one microtrap.

26. A method according to claim 23 wherein the vertical cell pairing (VCP) system further comprises a cover and a pair of side walls, such that the substrate, the cover and the pair of side walls together define a fluid passageway therebetween.

27. A method according to claim 26 wherein the at least one microtrap is secured to the substrate.

28. A method according to claim 26 wherein the at least one microtrap is secured to the cover.

29. A method according to claim 23 wherein the body of the at least one microtrap comprises a pair of surfaces disposed adjacent to the opening of the vertical slot, and further wherein the pair of surfaces are swept back relative to the opening of the vertical slot.

30. A method according to claim 29 wherein the pair of surfaces are swept back at an angle of between approximately 5 and approximately 35 degrees relative to the opening of the vertical slot.

31. A method according to claim 30 wherein the pair of surfaces are swept back at an angle of approximately 10 degrees.

32. A vertical cell pairing (VCP) system comprising:
a substrate having a top surface; and
at least one microtrap positioned adjacent the top surface of the substrate, the at least one microtrap comprising a body having a vertical groove and a vertical slot, wherein the vertical groove has an opening and an exit, the vertical groove being sized to seat a pair of cells therein when the pair of cells are vertically aligned, wherein the vertical slot has an opening and an exit, the vertical slot being sized to pass fluid therethrough but to prevent a cell from passing therethrough, wherein the exit of the vertical groove is in fluid communication with the opening of the vertical slot.

33. A method for vertically pairing cells, the method comprising:
providing a vertical cell pairing (VCP) system comprising:
a substrate having a top surface; and
at least one microtrap positioned adjacent the top surface of the substrate, the at least one microtrap comprising a body having a vertical groove and a vertical slot, wherein the vertical groove has an opening and an exit, the vertical groove being sized to seat a pair of cells therein when the pair of cells are vertically aligned, wherein the vertical slot has an opening and an exit, the vertical slot being sized to pass fluid therethrough but to prevent a cell from passing therethrough, wherein the exit of the vertical groove is in fluid communication with the opening of the vertical slot;
flowing a first slurry of cells over the top surface of the substrate and seating a first cell in the vertical groove of the at least one microtrap; and
flowing a second slurry of cells over the top surface of the substrate and seating a second cell in the vertical groove of the at least one microtrap.
